(19) United States
(12) Patent Application Publication
(10) Pub. No.: US 2025/0143833 A1
(43) Pub. Date: May 8, 2025

(54) PACKAGING FOR A MEDICAL PRODUCT

(71) Applicant: Aesculap AG, Tuttlingen (DE)

(72) Inventors: Christian Huber, Muehlheim (DE); Nadine Osswald, Tuttlingen (DE)

(21) Appl. No.: 18/730,231

(22) PCT Filed: Jan. 16, 2023
(86) PCT No.: PCT/EP2023/050889

§ 371 Date: Jul. 18, 2024

(30) Foreign Application Priority Data
Jan. 20, 2022 (DE) 10 2022 101 267.0

(51) Int. Cl. A61B 50/30

(52) U.S. Cl. CPC A61B 50/30; A61B 2050/314

(57) ABSTRACT

A packaging for or of a medical product includes a secondary packaging forming a sterile barrier and also defining a sealed or sealable receptacle for the medical product, and a product storage space and/or holder for contact-free storage of the product inside the secondary packaging. The product storage space and/or holder is in the form of a preferably pocket-like protective cover for receiving the medical product and for interspacing the secondary packaging and the product, the protective cover being made of a single, flexible flat cut that is produced by folding along at least one predefined hinged-fold line.

### CROSS-REFERENCE TO RELATED APPLICATIONS

This application is the United States national stage entry of International Application No. PCT/EP2023/050889, filed on Jan. 16, 2023, and claims priority to German Application No. 10 2022 101 267.0, filed on Jan. 20, 2022. The contents of International Application No. PCT/EP2023/050889 and German Application No. 10 2022 101 267.0 are incorporated by reference herein in their entireties.

### FIELD

The present disclosure relates to a packaging for or of a medical product (hereinafter also referred to as product protective packaging) comprising a secondary packaging or sterile packaging forming a sterile barrier, which forms a sealed or sealable receiving space for the medical product and a product storage space and/or holder for contact-free storage of the product within the secondary packaging.

### BACKGROUND

Medical products, in particular implants, are often inserted into the human or animal body. An important requirement for medical products, especially implants, is therefore that they are sterile in order to prevent infections from contaminated medical products in the human or animal body. To ensure that medical products are and remain sterile, they are sterilized or produced under sterile conditions, usually by the manufacturer. The sterile medical products are then introduced into sterile packaging (or secondary packaging). The sterile packaging has a sterile barrier that prevents the penetration of contaminants and an exchange between its interior and the environment. The sterile packaging, which encloses the medical product, is then vacuum-sealed. If the medical product is transported in the vacuumed sterile packaging, it can be ruled out that the medical product will be contaminated from outside the sterile packaging. If necessary, the medical product, in particular the implant, packaged and transported in this sterile manner can be unpacked from the sterile packaging and inserted into a human or animal body.

Vacuum-sealed sterile pouches are usually used as sterile packagings. In particular, if the medical product has a rough surface, which is particularly the case with hip and knee implants, there is a risk that the rough medical product surface will cause foil particles to detach from the sterile pouch or sterile packaging due to abrasion when the medical product is inserted into the sterile packaging and/or when the medical product is transported in the sterile packaging and/or when the medical product is removed from the sterile packaging. These foil particles can settle on the medical product and adhere to it. The foil particles remaining on the medical product can enter the body when the medical product is inserted into the animal or human body. This can (severely) affect the health and well-being of the patient concerned.

For medical products, especially those with a rough surface, a dimensionally stable/shape-retaining holder or product protective packaging, primarily made of (thermoset or thermoplastic) plastic, for example PETG, has therefore been provided, which is itself sterile and encloses the sterilized or sterile medical product. The product protective packaging with the medical product inside is then inserted into the vacuumable sterile packaging. Once the sterile packaging has been vacuumed, the medical product packaged in this way can be transported under sterile conditions. However, the dimensionally stable product protective packagings may break or bend due to the (strong) negative pressure that occurs during and as a result of vacuuming. In addition, dimensionally stable product protective packagings lead to the formation of many folds on the sterile pouch or sterile packaging that holds them.

### SUMMARY

It is thus an object of the present disclosure to reduce or eliminate the problems of the prior art. In particular, a packaging for a medical product (product protective packaging) is to be provided with a secondary packaging forming a sterile barrier, which forms a sealed or sealable, in particular vacuumized, receiving space for the medical product and a product storage space and/or holder for contact-free storage of the product within the secondary packaging, the product storage space of which does not break or become bent despite large forces acting on it. In addition, the corresponding product storage space should prevent the secondary packaging or sterile packaging receiving it, in particular vacuumed secondary packaging, from forming many folds.

This object is solved by the packaging (product protective packaging) according to the disclosure.

According to the disclosure, a packaging for or of a medical product, in particular a sterile medical product, preferably an implant with a rough surface structure (product protective packaging), is provided with a secondary packaging or sterile packaging forming a sterile barrier, which forms a sealed or sealable, in particular vacuumed, receiving space for the medical product and a product storage space and/or holder for contact-free storage of the product within the secondary packaging. The product storage space and/or holder is a, preferably pocket-shaped, protective cover for receiving the product and for interspacing the secondary packaging from the product, which protective cover is made from a single, flexible flat cut by folding (or flapping) along at least one predefined hinged fold line.

Such packaging has the advantage that the protective cover, in particular the pocket-shaped protective cover, which holds the medical product, protects it from foil particles in the secondary packaging or sterile packaging. In addition, the protective cover can withstand large forces, such as those present in the secondary packaging or sterile packaging during and as a result of vacuuming. The handling of the protective cover, in particular the insertion and removal of the protective cover into and from the secondary packaging or sterile packaging, is particularly easy and convenient. In principle, the medical product contained in the (pocket-shaped) protective cover can be easily inserted into the secondary packaging or sterile packaging with the aid of the protective cover, positioned therein and removed from the secondary packaging or sterile packaging.

Since the protective cover is configured from a flexible material, the protective cover does not break or bend irreversibly even under high pressure, e.g. due to the negative pressure during vacuuming of the secondary packaging or sterile packaging containing the protective cover. As the material of the protective cover is flexible, it adapts to the shape of the medical product it holds due to the negative pressure during vacuuming of the secondary packaging or sterile packaging and in the vacuumed secondary packaging or sterile packaging and only causes slight folding of the secondary packaging or sterile packaging. Since the protective cover is folded from a flat cut into a pocket shape, the protective cover is easy and inexpensive to manufacture. In the event that the flat cut is transferred into the, in particular pocket-shaped, protective cover exclusively by folding, no binding agents, such as adhesive, are required on the protective cover. This means that there is no risk of the binding agents detaching from the material of the protective cover and depositing on the medical product. The protective cover can also be unfolded again so that the medical product can be removed from the protective cover easily and with minimized friction. This minimizes the abrasion of particles from the material of the protective cover.

Advantageous aspects of the packaging according to the disclosure (product protective packaging) are explained below.

It is advantageous if the, in particular pocket-shaped, protective cover is closed on all sides toward the secondary packaging in a closed state created by folding.

In this way, the medical product arranged in the (pocket-shaped) protective cover cannot simply slip or fall out of the protective cover. This means that there is no risk of the medical product slipping into the secondary packaging, for example, where sterile packaging particles could then be deposited on the medical product due to abrasion. The protective cover, which is formed by folding the flat cut into a pocket shape, therefore ensures that the medical product it encloses does not accidentally fall or slip out of the protective cover at any time.

It is also advantageous if the single flat cut is made of several individual parts that are welded, sewn and/or glued together to form the single flat cut.

This has the advantage that the dimensions and shape of the flat cut can be individually and modularly adapted to the medical product to be accommodated in it.

It is particularly useful if, in the closed state, the opposite side edges of the base plate and the cover plate, in particular those lying on top of each other, are firmly bonded or form-fittingly connected to each other (apart from the side edges on which the hinged fold line is arranged).

In this way, each side of the protective cover can be easily and securely closed in the closed state. This prevents the medical product from escaping from one of the sides of the protective cover.

One embodiment of the protective cover relates to the flat cut comprising the following: a base plate serving as a storage space for the medical product and having a base latching device, a folding or cover plate (hereinafter also referred to simply as the cover plate) coupled to the base plate via a hinged fold line and can be brought into an operative engagement with the base latching device in a position where the cover plate is folded toward/onto the base plate to maintain the folded position, and a first side plate coupled to the base plate via a swivel fold line aligned perpendicular to the hinged fold line and having a side latching device for holding the first side plate in a pivoted position toward the base plate.

If the protective cover has a base plate on which a cover plate is folded in the closed state (i.e. on which the cover plate rests), the base plate and cover plate cooperate to form a main compartment for accepting a medical product. In this way, the protective cover can be configured simply by folding a flat cut with two sides, namely cover plate and base plate. The side on which the cover plate and base plate have the common side edge and thus the hinged fold line between them, the pocket-shaped protective cover is always closed. The side plate can be used to close another side edge of the base plate and, if necessary, the cover plate. The operative engagement between the base latching apparatus of the base plate and the cover plate is used to connect the corresponding side edges of the base plate and cover plate to each other and thus close them.

It is also possible for the base plate to have two receptacle slits that run parallel to each other and are spaced apart. These can be configured to cooperatively attach a medical product or packaging means, e.g. a drying agent, to the base plate.

If, for example, a medical product is placed loosely on the base plate and a product agent is fixed in the receptacle slits, the receptacle slits prevent the product from changing its position during transportation of the protective cover inside the protective cover.

In addition, it may be provided that the flat cut further comprises a second side plate which is coupled to the base plate opposite the first side plate via a swivel fold line aligned perpendicular to the hinged fold line and has a side latching device for holding the second side plate in a position pivoted toward the base plate.

The second side plate can be used to close all sides of the protective cover. This is because in the closed state, the hinged fold line closes one side of the protective cover, in particular one end of the protective cover. The base latching device is advantageously located on the side of the protective cover opposite the hinged fold line in the closed state, in particular the other end face of the protective cover. The operative engagement between the base latching device of the base plate and the cover plate thus closes the other end face of the protective cover. The other two sides of the protective cover are closed by the side plates.

In particular, it is provided that the respective side-engaging device of each of the first side plate and the second side plate is configured as a latching slit, via which the first side plate and the second side plate can be hooked together, or the respective side-engaging device of the first side plate and the second side plate is configured as a receptacle, in particular a slit-like receptacle, into which a tab-like portion of the folding or cover plate can be inserted.

By hooking or via the receptacle, the first and second side plates can be form-fittingly connected to each other or to the cover plate in a particularly secure manner. This prevents the medical product from simply slipping or falling out of the protective cover folded into the closed state.

It is advantageous, in particular, if the first and second side plates each have an overlapping region with which they overlap each other in the closed state. The latching slit can then be provided in the overlapping region of each side plate, wherein the latching slit of one side plate is provided to be interlocked with the latching slit of the overlapping region of the other side plate in the closed state. As an alternative to the latching slit, the receptacle is provided in the overlapping region of a side plate, which receptacle in the closed state lies above the receptacle of the overlapping region of the other side plate and forms a continuous receptacle for receiving the tab-shaped portions of the cover plate.

In both ways, the side plates can be securely connected to each other and kept in a closed state to prevent accidental opening.

According to another embodiment of the protective cover, the flat cut comprises the following: a base plate serving as a storage space for the medical product and having a base latching device, a folding or cover plate coupled to the base plate via a hinged fold line and can be brought into operative engagement with the base latching device in a position where the cover plate is folded toward/onto the base plate to maintain the folded position, a closing tab coupled to the base plate or the folding or cover plate via a swivel fold line aligned parallel to the hinged fold line, and an insert tab which is coupled via a swivel fold line aligned parallel to the hinged fold line to that out of the base plate and folding or cover plate which is not coupled to the closing tab, and which forms between itself and the base plate or folding or cover plate coupled thereto a, in particular slit-like, tab receptacle for receiving the closing tab.

This version of the protective cover is particularly simple. This is because side plates are not required here to close the side edges of a pair of side edges. Rather, two side edges of the base plate and cover plate that are opposite each other in the closed state, in particular lying on top of each other, are closed to the outside by the operative engagement of the base latching device and the cover plate. The side edges of the base plate and the cover plate, which are parallel to the hinged fold line in the closed state, are connected to each other via the closing tab that engages in the tab receptacle. In the closed state, the closing tab form-fittingly engages in the tab receptacle or engages behind it. The other side edges that are opposite each other in the closed state, in particular those that lie on top of each other, can each be firmly bonded to each other, e.g. by welding, sealing or gluing. Further tabs or the like can therefore be omitted on these side edges. This makes the flat cut very simple and therefore easy and inexpensive to manufacture.

In another embodiment of the protective cover, the flat cut comprises a base plate serving as a storage space for the medical product and having a base latching device, a folding or cover plate which is coupled to the base plate via a hinged fold line and can be brought into operative engagement with the base latching device in a position where the cover plate is folded onto the base plate in order to hold the folded position, and a first additional tab each on the base plate and on the folding or cover plate, which tabs are adjacent to the side edges of the base plate and of the folding or cover plate which are adjacent to one another and extend perpendicularly to the hinged fold line and, in the closed state, together form an additional compartment for receiving a further product, in particular a further medical product or a packing means accompanying a medical product.

Advantageously, the additional compartment is spatially separated from the main compartment formed by the cover plate and base plate in the closed state (by a firmly bonded region). This means that a further medical product or a packing means to be transported with the medical product, e.g. a drying agent, can be arranged in one and the same pocket-shaped protective cover with spatial separation. If, for example, two medical products, which in particular each have a rough surface, are arranged in the pocket-shaped protective cover in this way, it is possible to prevent the medical products from repelling each other and thereby removing material from each other.

It is advantageous if the flat cut also has further second additional tabs, in particular two additional tabs each, on the base plate and on the folding or cover plate, which adjoin a side edge of the first additional tab which is spaced parallel to the side edge of the base plate or the folding or cover plate extending perpendicular to the hinged fold line, and the second additional tabs on the base plate or the folding or cover plate are spaced apart from one another and, in the closed state, together with the second additional tab lying above or below it, form further additional compartments for receiving further products on the other of the base plate and the folding or cover plate.

In this way, several medical products can be transported separately from each other in one and the same pocket-shaped protective cover.

In another embodiment of the protective cover, the flat cut has the following features:
A base plate serving as a storage space for the medical product, which base plate has a base latching device,
a folding or cover plate which is coupled to the base plate via the hinged fold line and can be brought into operative engagement with the base latching device a position where the cover plate is folded toward/onto the base plate to hold the folded position,
wherein the folding or cover plate is divided into a connecting portion and a tear-open portion, which is separated from the connecting portion by at least one perforation line forming a predetermined breaking point between the connecting portion and the tear-open portion.

When such a flat cut is transferred to the closed pocket shape of the protective cover, the perforation line (as a predetermined breaking point) enables the tear-open portion to be torn open and thus be separated from the connecting portion, which (in the protective cover state of the closed pocket shape) remains fixedly and firmly bonded to the base plate. By simply applying a tensile force to the tear-open portion, it can be detached from the connecting portion and thus also from the remaining folding or cover plate, so that an opening for removing a medical product contained in the protective cover can be exposed quickly and easily.

Preferably, the (long) side edges of the folding or cover plate and the base plate, which are opposite each other in the folded position and extend perpendicularly from the hinged fold line, can be firmly bonded to each other or are bonded to each other in a state in which the protective cover has a closed pocket shape.

Preferably, in the case of only one perforation line, this one perforation line completely separates the tear-open portion from the connecting portion. In the case of two perforation lines, the two perforation lines enclose the tear-open portion between them and in this way (completely) separate it from the connecting portion. In this way, the perforation line(s) clearly and easily separate(s) the tear-open portion from the connecting portion.

In particular, in the case of two perforation lines, the two perforation lines are arranged on the folding or cover plate in such a way that the tear-open portion divides the connecting portion into two halves (possibly of equal size) that are separated from each other. In other words, the tear-open portion is arranged between the two halves of the connecting portion.

It may be provided that a first end of the at least one perforation line and a second end of the same perforation line or, in the case of two perforation lines on the same folding or cover plate, a first perforation line and a second perforation line open into a (short) side edge of the folding or cover plate opposite the hinged fold line. In other words, the tear-open portion is completely limited by the at least one perforation line and by the side edge of the folding or cover plate opposite the hinged fold line.

It is also useful if, starting from the side edge of the folding or cover plate opposite the hinged fold line, a tab-like portion of the folding or cover plate extends away from the folding or cover plate, which in particular can be brought into operative engagement with the base latching device, preferably designed as a tab receptacle, on the base plate, and which is integrally configured with the tear-open portion. The tab-like portion of the cover plate or base plate can be easily gripped by a user. By applying a pulling or tearing force to the tab-like portion, the user can easily and quickly remove the tear-open portion along the perforation line from the connecting portion and thus from the rest of the folding or cover plate (so that an opening for removing a medical product held in the protective cover can be easily and quickly exposed), preferably with just one hand movement.

It is also conceivable that the first end of the at least one perforation line and the second end of the same perforation line or, in the case of two perforation lines on the same folding or cover plate, the first perforation line and the second perforation line are spaced apart by the width of the tab-like portion at the point at which they each open into the (short) side edge of the folding or cover plate opposite the hinged fold line. This means that the tear-open portion merges directly (without offset or step) into the tab-like portion. This makes it easier to remove or tear open the tear-open portion and produce the flat cut of the corresponding protective cover.

It is also conceivable that the connecting portion is integrally bonded/configured to the base plate. In this case, the connecting portion is directly adjacent to the base plate with a hinged fold line in between.

It is also useful if the tear-open portion is integrally connected to the connecting portion. This facilitates the production and handling of an appropriately formed flat cut of the protective cover.

In addition, it may be provided that two perforation lines are provided which are configured non-linearly (i.e. with at least one curvature/curve) and are arranged axially symmetrically to one another with respect to a line bisecting the hinged fold line and wherein each one extends continuously from the hinged fold line to the side edge of the folding or cover plate opposite the hinged fold line. This variant has the advantage that such a packaging or protective cover is particularly easy to manufacture. In addition, the non-linear design of the perforation lines, i.e. perforation lines without tips within the course of the perforation lines, has the advantage that the tear-open portion can be separated from the connecting portion particularly effectively and without leaving any residue.

In addition, it may be provided that the base latching device of the base plate is configured as a, in particular slit-like, receptacle which is arranged in the immediate vicinity of one of the side edges of the base plate, and the folding or cover plate has a tab-shaped portion which extends from the side edge of the folding or cover plate, which in the closed state is opposite to the side edge next to the receptacle of the base plate, and which is insertable into the receptacle of the base plate, or it is possible that the base latching device of the base plate is configured as a tab-shaped portion of the base plate and the folding or cover plate has a receptacle which in the closed state receives this tab-shaped portion of the base plate.

In other words, the base latching device is designed as a receptacle in the base plate or as a tab-like portion of the base plate, which is configured complementary to a cover plate engagement device (tab-like portion or receptacle) provided on the cover plate. In this way, the operative engagement between base plate and cover plate can be easily realized.

The side edges of the base plate and the cover plate can be connected quickly and easily using the tab-like portion of the cover plate or base plate that engages in the receptacle. The tab-like portion of the cover plate or base plate can also be easily gripped by a user.

Advantageously, the entire flat cut that forms the protective cover is integrally configured. This means that all tabs or the tab-like portion of the cover plate or base plate, which are located on the flat cut, are integrally configured with it (and therefore also made of the same material).

It is also helpful if the tab-like portion of the cover plate or base plate is stabilized by an accumulation of material, i.e. by a thicker wall thickness than the rest of the protective cover, preferably double the wall thickness.

If the tab-like portion of the cover plate or base plate is stabilized in this way, the tab-like portion of the cover plate or base plate can be gripped easily and it is very unlikely that the tab-like portion of the cover plate or base plate will tear or become detached.

It is advantageous if the tab-like portion of the cover plate or base plate protrudes outward from the side edge (side edge of the cover plate or base plate) supporting it and, in particular at its rounded end, has a recess, in particular a circular, round or angular recess.

The recess makes it easier for a user to grip the tab-like portion of the cover plate or base plate. As the tab-like portion of the cover plate or base plate protrudes outward from the side edge supporting it, the tab-like portion of the cover plate or base plate is particularly easy to grip.

The tab-shaped portion of the cover plate or base plate can be tongue-shaped or configured in the form of a loop.

In the embodiment in which the cover plate is divided into the connecting portion and the tear-open portion, it is particularly advantageous if the tab-shaped portion is integrally, i.e. rigidly bonded to the tear-open portion. In this way, the tear-open portion can be gripped particularly easily by the tab and be separated from the connecting portion (and thus from the base plate firmly bonded to the connecting portion).

It is conceivable that the protective cover or the flat cut forming it (the protective cover) is made of a flexible, abrasion-resistant and sterilizable material, preferably TPU, Tyvek, silicone or plastics with comparable properties.

It is also conceivable that the material from which the protective cover or the flat cut forming it is made is an extruded or blown film, a mesh, a fabric or a nonwoven (whereby the material may have thermoformed elements in places). It can also be a mono-material or a composite material.

If the protective cover is configured from such a material, the protective cover is ideally suited for the receptacle of, in particular, sterile medical products.

Furthermore, it is conceivable that the side edge of the base plate or cover plate, in the immediate vicinity of which the receptacle extending at least in sections along this side edge has an outward projecting, in particular curved, projection. This projection can be easily gripped by a user and simplifies handling of the protective cover, in particular when opening the pocket-shaped protective cover.

### BRIEF DESCRIPTION OF THE DRAWINGS

FIG. 1 is a top view of a flat cut of a protective cover according to a first embodiment;

FIG. 2A is a top view of the flat cut from FIG. 1 with the products arranged on it;

FIG. 2B is a top view of the flat cut from FIG. 2A formed into the pocket shape;

FIG. 3A is a pictorial representation of the process steps for folding the pocket-shaped protective cover from the flat cut in FIG. 1;

FIG. 3B is a pictorial representation of the process steps for removing the medical product from the protective cover assembled according to FIG. 3A;

FIG. 4 is a top view of a flat cut of a protective cover according to a second embodiment;

FIG. 5A is a top view of the flat cut from FIG. 4 with the products arranged on it;

FIG. 5B is a top view of the flat cut from FIG. 5A formed into the pocket shape;

FIG. 6 is a top view of a flat cut of a protective cover according to a third embodiment;

FIG. 7A is a top view of the flat cut from FIG. 6 with the product arranged on it;

FIG. 7B is a top view of the flat cut from FIG. 7A formed into the pocket shape;

FIG. 8 is a top view of a flat cut of a protective cover according to a fourth embodiment;

FIG. 9A is a top view of the flat cut from FIG. 8 with products arranged on it;

FIG. 9B is a top view of the flat cut from FIG. 9A formed into the pocket shape;

FIG. 10 is a top view of a flat cut of a protective cover according to a fifth embodiment;

FIG. 11A is a top view of the flat cut from FIG. 10 with products arranged on it;

FIG. 11B is a top view of the flat cut from FIG. 11A formed into the pocket shape;

FIG. 12 is a top view of a flat cut of a protective cover according to a sixth embodiment;

FIG. 13A is a top view of a protective cover folded from the flat cut in FIG. 12;

FIG. 13B is a perspective view of the protective cover from FIG. 13A, into which a medical product is inserted;

FIG. 13C is a perspective view of the protective cover from FIG. 13B, which is closed to the pocket shape and encloses the medical product;

FIG. 14 is a pictorial representation of the process steps for removing the medical product from the protective cover assembled according to FIG. 13A to FIG. 13C;

FIG. 15A to FIG. 15L show a first to twelfth variant of the protective cover 1 of the sixth embodiment;

FIG. 16A is a pictorial representation of the process steps for inserting a medical product into a packaging according to the disclosure; and

FIG. 16 is a pictorial representation of the process steps for unpacking a medical product from the packaging according to the disclosure.

### DETAILED DESCRIPTION

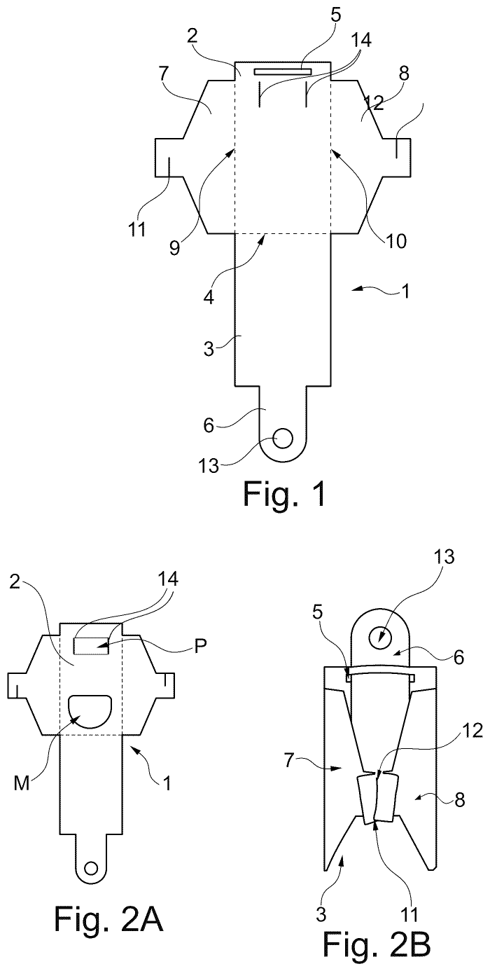

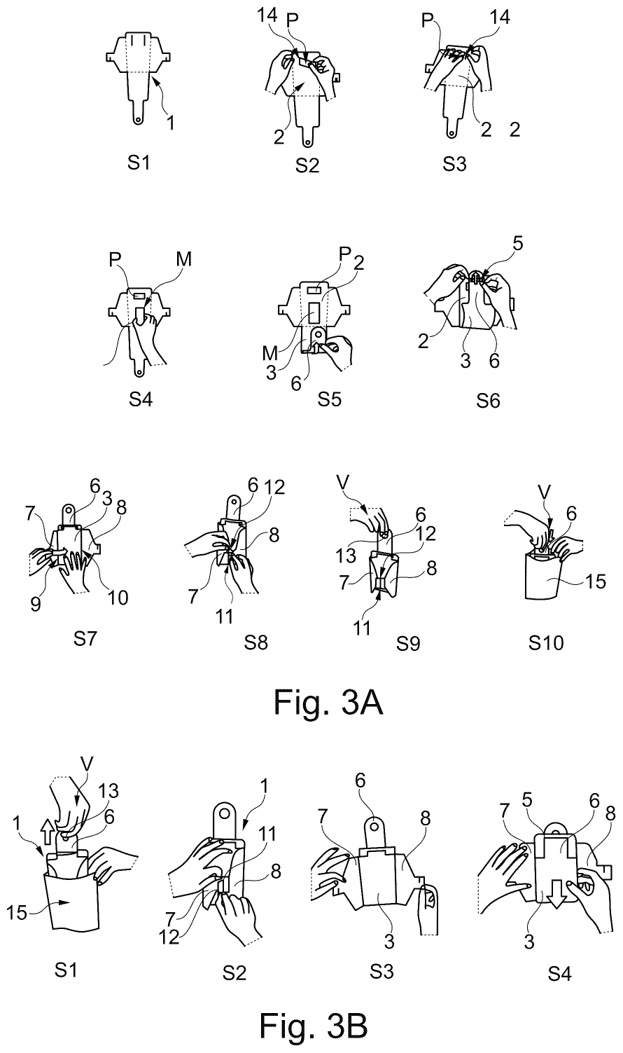

FIG. 1 is a top view of a flat cut of a protective cover 1 of a first embodiment. The flat cut is configured from a flexible material. The protective cover 1 is used for receiving a medical product (not shown in FIG. 1), in particular a medical product to be sterilized, preferably an implant with a rough surface structure. The protective cover 1 is formed from the flexible flat cut by folding the flat cut into a pocket shape (see FIG. 3A). The protective cover 1 is provided to and configured in its dimensions and shape to be arranged in a closed state accommodating the medical product in a secondary packaging (not shown here) or sterile packaging (hereinafter simply referred to as sterile packaging), in particular one that can be vacuumed.

The flat cut of the protective cover 1 has a substantially rectangular base plate 2 with two opposing short side edges and two opposing long side edges. A cover plate 3 with a rectangular portion adjoins a short side edge of the base plate 2. The rectangular portion of the cover plate 3 also has two opposing short side edges and two opposing long side edges. A hinged fold line 4 along the meeting (and coinciding) short side edges of base plate 2 and cover plate 3 marks the transition or boundary between base plate 2 and cover plate 3. The cover plate 3 can be folded over/onto the base plate 2 relative to the base plate 2 via the hinged fold line 4. The long side edges of the base plate 2 are advantageously longer than the long side edges of the cover plate 3. On the other short side edge of the base plate 2, which faces away from the cover plate 3 and is (parallel) opposite the hinged fold line 4, the base plate 2 (within its rectangular shape) has a receptacle 5 as a base latching device. The receptacle 5 is located directly below the corresponding short side edge. The receptacle 5 is shaped into an elongated slit. The receptacle 5 extends parallel to the short side edge of the base plate 2. From the shorter side edge of the rectangular portion of the cover plate 3, which faces away from the base plate 2, a tab-like portion (hereinafter simply referred to as ‘tab 6’) of the cover plate 3 extends outward, in particular integrally formed with the cover plate 3. The cover plate 3 therefore has a rectangular portion and a tab-shaped portion. The dimensions of tab 6 and receptacle 5 are configured relative to each other in such a way that tab 6 can be inserted into receptacle 5. This means that the receptacle 5 is at least as wide as the tab 6.

A first side plate 7 and a second side plate 8 each adjoin the opposing long side edges of the base plate 2. The lower edge of the first side plate 7 and the second side plate 8 extends at the level of the hinged fold line 4. Overall, the first and second side plates 7 and 8 do not extend over the entire length of the long side edges of the base plate 2. The common side edge between the first side plate 7 and the base plate 2 is configured as a swivel fold line 9. The common side edge between the second side plate 8 and base plate 2 is configured as a swivel fold line 10. The first and second side plates 7, 8 can be folded over the base plate 2 or the cover plate 3 relative to the base plate 2 via the respective swivel fold line 9, 10. The first and second side plates 7, 8 each have a latching slit 11, 12 at their overlapping regions that overlap in the closed state of the protective cover 1. The latching slits 11, 12 are each realized by a cut in the overlapping regions of the respective first or second side plate 7, 8. The latching slit 11 of the first side plate 7 (which runs parallel to the swivel fold line 9) extends from the lower edge of the overlapping region of the first side plate 7 to its center. The latching slit 12 of the second side plate 8 (which runs parallel to the swivel fold line 10) extends from the upper edge of the overlapping region of the second side plate 8 to its center. Of course, the latching slits 11, 12 could be arranged exactly the other way round in the respective overlapping regions. The latching slits 11, 12 are intended to form-fittingly engage or interlock with each other in the closed state.

It can also be seen that tab 6 is narrower than the short side edge of cover plate 3. The tab 6 is positioned centrally relative to the short side edge. The tab 6 has a recess 13 at its rounded free end. The recess 13 is circular. The recess 13 serves as a gripping aid. Below the slit-like receptacle 5, the base plate 2 has at least two slits 14 that are spaced apart and run parallel to each other and to the long side edges of the base plate 2. For example, a medical product or a packaging means accompanying the medical product, e.g. a drying agent, can be fixed in the slits 14.

FIG. 2A shows the flat cut of the protective cover 1 known from FIG. 1 with a medical product M arranged on the base plate 2. A packing means P, e.g. a drying agent, is fixed between the two slits 14 on the base plate 2.

FIG. 2B shows the protective cover 1 folded from the flat cut of the protective cover 1 of FIG. 2A. In other words, the closed state of the protective cover 1 of the first embodiment is shown in FIG. 2B. The cover plate 3 is folded over the base plate 2 along the hinged fold line 4, which is not visible here. The tab 6 is inserted into the slit-like receptacle 5 on the base plate 2. The base plate 2 and cover plate 3 thus enclose the medical product M and the packaging means P. The first and second side plates 7, 8 are each folded over the cover plate 3 via the hinged fold lines 9, 10, which are not visible here. The latching slits 11, 12 of the overlapping regions of the respective first and second side plates 7, 8 interlock and close the protective cover 1. In this way, the protective cover 1 is closed on all sides. The medical product M and the packing means P thus remain in the protective cover 1 even in the event of shocks during transportation.

FIG. 3A shows the process steps S1 to S10 for folding the pocket-shaped protective cover 1 from the flat cut in FIG. 1. Step S1 shows the spread-out flat cut of the protective cover 1. In step S2, one (short) side of a packing means P is inserted into one of the slits 14 in the base plate 2. In step S3, the side of the packing means P opposite the side that was inserted into one slit 14 in step S2 is inserted into the other slit 14. This fixes the packing means to the base plate 2. In step S4, a medical product M is positioned on the base plate 2. In step S5, the cover plate 3 is gripped by the tab 6 and folded over the base plate 2. In step S6, the tab 6 of the cover plate 3 is inserted into the receptacle 5 of the base plate 2. The protective cover 1 is thus closed on its underside (via the continuous hinged fold line 4) and on its upper side (via the tab 6 engaging in the receptacle 5). In step S7, the first side plate 7 is folded over the cover plate 3 along the swivel fold line 9 and the second side plate 8 is folded over the cover plate 3 along the swivel fold line 10. In step S8, the latching slits 11, 12 are hooked into each other, thus fixing the first and second side plates 7, 8. The protective cover 1 is thus also closed at the sides. In step S9, a user V holds the protective cover 1 folded to the pocket shape and holding the medical product M and the packing means P, using the recess 13 as a gripping aid. In step S10, a user V packs the protective cover 1 prepared in steps S1 to S9 into a sterile packaging 15 using the recess 13 as a gripping aid (see direction of arrow), thus forming an embodiment of the packaging according to the disclosure.

FIG. 3B schematically shows process steps S1 to S4 for removing the medical product M from the pocket-shaped protective cover 1 folded according to FIG. 3A. In step S1, the protective cover 1 is pulled out of the sterile packaging 15 (see direction of arrow) via the tab 6, in particular via the recess 13 as a gripping aid. In step S2, the interlocking latching slits 11, 12 are unhooked. In step S3, the first and second side plates 7, 8 are folded outward from the cover plate 3 and thus are unfolded. In step S4, the tab 6 is pulled out of the receptacle 5 and the cover plate 3 is folded away from the base plate 2. After step S4 has been carried out, the medical product M and packing means P lie open on the base plate 2 and can be removed from the protective cover 1, which has been unfolded to form a flat cut (not shown here).

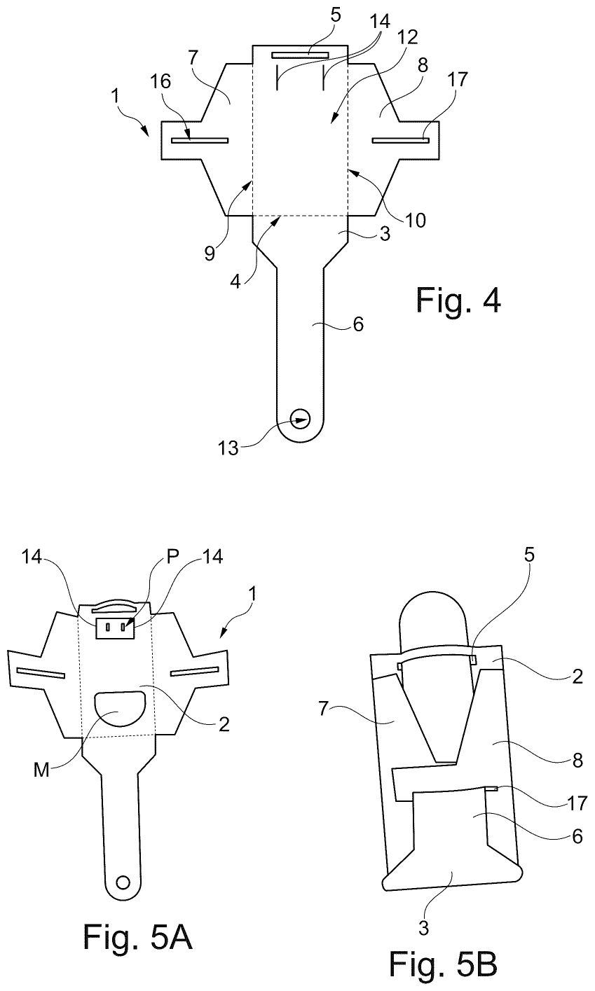

FIG. 4 is a top view of a flat cut of a protective cover 1 of a second embodiment. The flat cut of the protective cover 1 of the second embodiment substantially corresponds to the flat cut of the protective cover 1 of the first embodiment. The base plate 2 of the protective cover 1 of the second embodiment is constructed in exactly the same way as the base plate 2 of the protective cover 1 of the first embodiment (see FIG. 1). Here too, a first and a second side plate 7, 8 are arranged on the base plate 2, the dimensions of which are substantially the same as in the first embodiment. In contrast to the first embodiment, the first and second side plates 7, 8 each have a slit-like receptacle 16, 17 in their overlapping regions instead of the latching slits 11, 12. The slit-like receptacles 16, 17 each correspond substantially in shape and dimensions to the receptacle 5 on the base plate 2.

The side edges of the rectangular portion of the cover plate 3, which continue the long side edges of the base plate 2, are considerably shorter than the long side edges of the base plate 2 and even considerably shorter than the, i.e. shorter than half of the, short side edge(s) of the rectangular portion of the cover plate 3 or of the base plate 2 (or than the hinged fold line 4). On the other hand, the tab 6 of the cover plate 3 is considerably longer than that of the protective cover 1 of the first embodiment.

FIG. 5A shows a top view of the flat cut from FIG. 4. Here, a medical product M is arranged on the base plate 2. In addition, a packing means P is inserted into the slits 14 on the base plate 2.

FIG. 5B shows the protective cover 1 folded from the flat cut of the protective cover 1 of FIG. 5A. In other words, the closed state of the pocket-shaped protective cover 1 of the second embodiment is shown in FIG. 5B. The first side plate 7 rests on the base plate 2. The second side plate 8 lies on the base plate 2 and on the first side plate 7. The overlapping region of the second side plate 8 overlaps the overlapping region of the first side plate 7 from above. More precisely, the receptacle 17 of the second side plate 8 lies exactly above the receptacle 16 of the first side plate 7. Thereby, the receptacle 16 and the receptacle 17 of the respective first and second side plates 7, 8 form a continuous receptacle for receiving the tab 6. The cover plate 3 is folded over the first and second side plates 7, 8 and rests on them. The tab 6 is inserted into the superimposed receptacles 16, 17 of the first and second side plates 7, 8 and is inserted into receptacle 5 of the base plate 2. The protective cover 1 of the second embodiment, like that of the first embodiment, is also closed on all sides in the folded pocket shape.

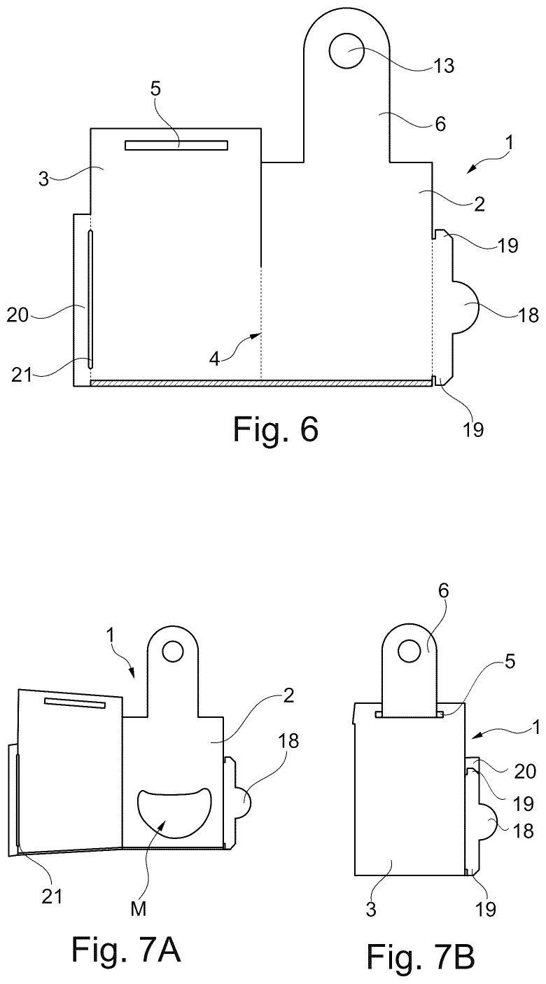

FIG. 6 is a top view of a flat cut of a protective cover 1 of a third embodiment. Here, the long side edges of the base plate 2 and the cover plate 3 coincide and form the hinged fold line 4 at this point, at least in sections. The hinged fold line 4 does not extend over the entire length of the long side edges of the base plate 2 or cover plate 3. The hinged fold line 4 only extends from the lower short side edge of the base plate 2 or cover plate 3 to approximately half of the corresponding long side edge. In the remaining region (the upper half) of the long side edges of the base plate or cover plate 3, these do not coincide and are configured separately from each other (and are not connected to each other). In the closed state of the protective cover 1, the protective cover 1 is therefore open in these regions. In this embodiment, in contrast to the first two embodiments, the base plate 2 (like the cover plate 3 in the first two embodiments) is provided with a rectangular portion and a tab-shaped portion. At the upper short side edge of the rectangular portion in FIG. 4, the base plate 2 has the tab-shaped portion or the tab 6. Here too, the tab 6 is provided with a circular recess 13. In the region of its upper short side edge, the cover plate 3 has the receptacle 5 running parallel to this side edge and spaced apart from it. Accordingly, the long side edge of the base plate 2 is slightly shorter than the long side edge of the cover plate 3, which has the receptacle 5. More precisely, the long side edge of the base plate 2 ends below the receptacle 5 of the cover plate 3.

On the long side edge opposite the hinged fold line 4, the base plate 2 has a closing tab 18 extending at least partially over the long side edge. The closing tab 18 is pivotable relative to the base plate 2 via a swivel fold line running parallel to the hinged fold line 4. The closing tab 18 has a barbed hook 19 at each of its two longitudinally opposite ends. On the long side edge opposite the hinged fold line 4, the cover plate 3 has an insert tab 20 extending at least partially along the long side edge. The insert tab 20 is pivotable relative to the cover plate 3 via a swivel fold line running parallel to the hinged fold line 4. Closing tab 18 and insert tab 20 are configured integrally with the cover plate 3 and the base plate 2 in particular. A slit-like tab receptacle 21 is configured between the cover plate 3 and the insert tab 20 in the region of the long side edge along this side edge. In other words, the tab receptacle 21 interrupts or replaces the long side edge at this point. The closing tab 18 and the insert tab 20 are attached at the same height, so that in the closed state the closing tab 18 can engage with the barbed hook 19 in the tab receptacle 21 or can be inserted into it. The length of the closing tab 18 is, in particular exactly, for the length of the barbed hook 19 longer than the tab receptacle 21.

The lower short side edges of base plate 2 and cover plate 3 are firmly bonded together, as shown by the hatched region. This means that base plate 2 and cover plate 3 are bonded, welded or sealed together in the shaded region.

FIG. 7A shows a top view of the flat cut of the protective cover 1 from FIG. 6 with medical product M arranged on it. More precisely, the medical product M lies on the base plate 2.

FIG. 7B is a top view of the flat cut from FIG. 7A formed into the protective cover 1. In other words, the closed state of the pocket-shaped protective cover 1 of the third embodiment is shown in FIG. 7B. The cover plate 3 is folded over the base plate 2 and thus covers the medical product M. The tab 6 of the base plate 2 is inserted into the receptacle 5 on the cover plate 3. The closing tab 18 is inserted into the tab receptacle 21. The barbed hooks 19 prevent the closing tab 18 from coming loose from the tab receptacle 21. In other words, the barbed hooks 19 engage behind the tab receptacle 21. Thereby, a form-fitting connection between the closing tab 18 and the insert tab 20 is formed. The lower short side edges of base plate 2 and cover plate 3 are firmly bonded together (welded, glued or sealed) in the closed state shown here.

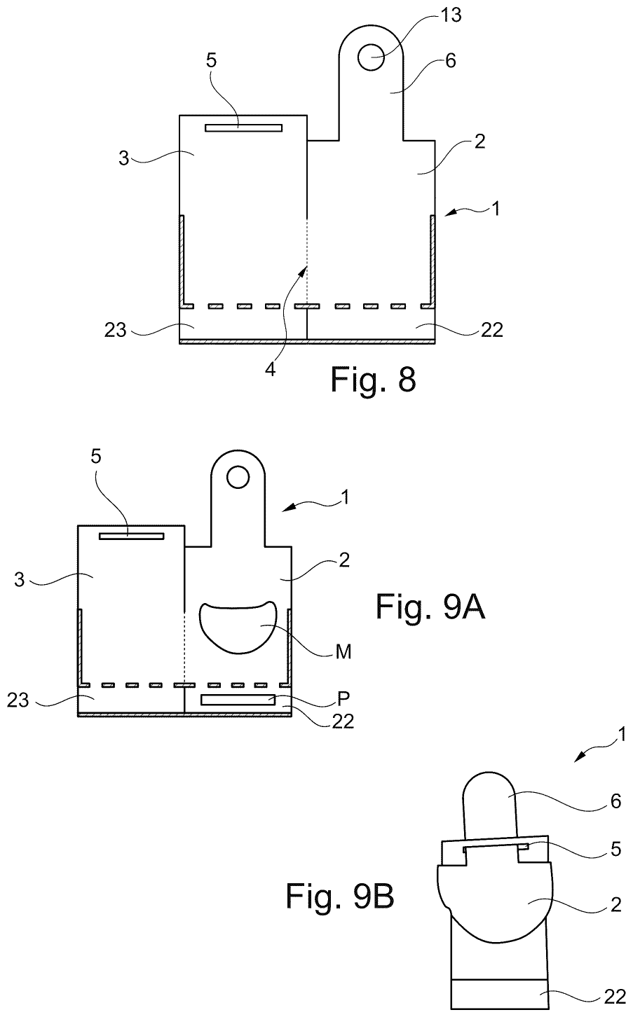

FIG. 8 shows a top view of a flat cut of a protective cover 1 of a fourth embodiment. The protective cover 1 of the fourth embodiment corresponds substantially to that of the third embodiment. However, in the protective cover of the fourth embodiment according to FIG. 8, the closing tab 18 and also the insert tab 20 are omitted. Instead, the long side edges of base plate 2 and cover plate 3 in the region facing away from the short side edges, which are provided with receptacle 5 and tab 6 respectively, have regions for firmly bonded connection (see hatched regions). The hatched regions along the short side edges are interrupted. This means that separate points or regions of the lower short side edges are firmly bonded together.

Furthermore, a first additional tab 22, in particular integrally formed with the base plate 2, adjoins the lower side edge of the base plate 2 and a first additional tab 23, in particular integrally formed with the cover plate 3, adjoins the lower side edge of the cover plate 3. The first additional tab 22 and the first additional tab 23 are provided with regions for a firmly bonded connection (see hatched region) on their side edge facing away from or being opposite of the short side edge of the base plate 2 or cover plate 3. In the closed state, the first additional tab 23 rests on the first additional tab 22 and is firmly bonded to it at the points of the hatched regions. The first additional tab 23 thus forms an additional compartment in cooperation with the first additional tab 22, which is spatially separated from the main compartment defined by the base plate 2 and the cover plate 3. In the additional compartment, a further medical product or packing means can therefore be arranged separately from a medical product and possibly packing means in the main compartment.

FIG. 9A shows a top view of the flat cut of the protective cover 1 in FIG. 8. A medical product M is arranged on the base plate 2. This is then arranged in the closed state in the main compartment. Furthermore, a packing means P is arranged on the first additional tab 22, which is adjacent to the base plate 2. This is then arranged in the additional compartment in the closed state.

FIG. 9B is a top view of the flat cut from FIG. 9A formed into the protective cover 1. In other words, the closed state of the protective cover 1 of the fourth embodiment is shown in FIG. 9B. In this representation, the base plate 2 is facing the viewer. Cover plate 3 and base plate 2 together form the main compartment in which the medical product M is arranged. The side edges of cover plate 3 and base plate 2 are firmly bonded together in the hatched regions shown in FIG. 8. The tab 6 of the base plate 2 is inserted into the corresponding receptacle 5 of the cover plate 3. Together with the first additional tab 22, the first additional tab 23 forms the additional compartment in which the packing means P is arranged. The lower side edges of the first additional tabs 22, 23 are firmly bonded together in the hatched region (see FIG. 8). The firmly bonded connection in the region between base plate 2 or cover plate 3 and the first additional tabs 22, 23 spatially separates the main compartment and the additional compartment from one another. The main compartment is closed on all sides. The additional compartment is open at its wide (short) side edges.

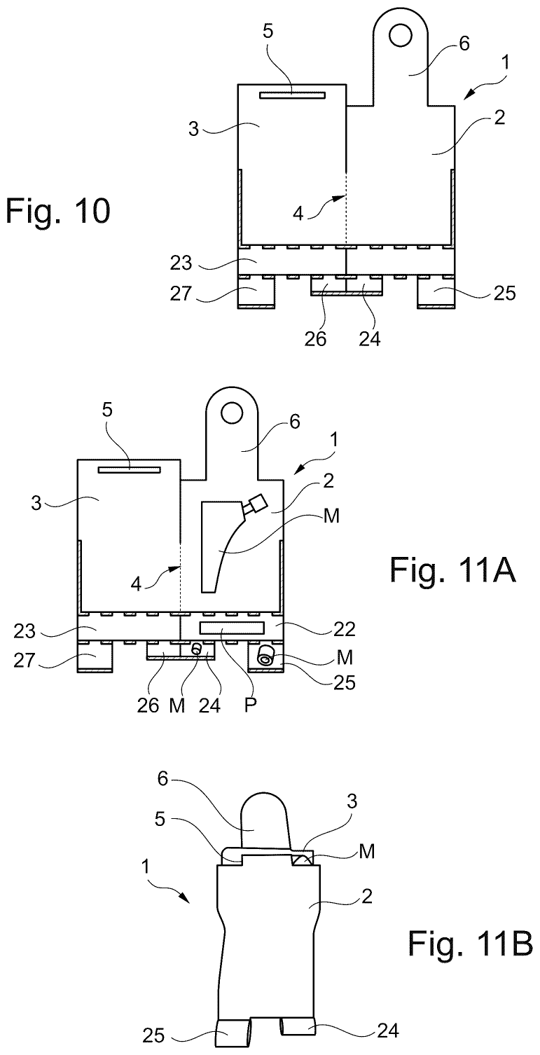

FIG. 10 is a top view of a flat cut of a protective cover 1 of a fifth embodiment. The protective cover 1 of the fifth embodiment substantially corresponds to the protective cover 1 of the fourth embodiment. In contrast to the protective cover 1 of the fourth embodiment, that of the fifth embodiment has two spaced-apart second additional tabs 24, 25 below the first additional tab 22 of the base plate 2. The second additional tab 24 is smaller than the second additional tab 25. The outer side edge of the second additional tab 24 continues the hinged fold line 4. The outer side edge of the second additional tab 25 continues the long side edge of the base plate 2 opposite the hinged fold line 4. The inner side edges of the second additional tab 24 and the second additional tab 25 face each other. Mirroring this, with the hinged fold line 4 as the center line, there are two second additional tabs 26, 27 spaced apart from each other below the first additional tab 23 of the cover plate 3. The second additional tab 26 is smaller than the second additional tab 27. The outer side edge of the second additional tab 26 continues the hinged fold line 4 and thus coincides with the outer side edge of the second additional tab 24. The outer side edge of the second additional tab 27 continues the long side edge of the cover plate 3 opposite the hinged fold line 4. The inner side edges of the second additional tab 26 and the second additional tab 27 face each other.

In the closed state, the second additional tab 24 cooperates with the second additional tab 26 so that the second additional tab 26 rests on the second additional tab 24 (covering the entire surface) and forms an additional compartment. The second additional tab 24 and the second additional tab 26 are therefore of the same size. In the closed state, the second additional tab 25 also cooperates with the second additional tab 27, so that the second additional tab 27 rests on the second additional tab 25 (covering the entire surface) and forms a further additional compartment. The second additional tab 25 and the second additional tab 27 are therefore of the same size. To form the additional compartments, the respective lower side edges of the second additional tabs 24 to 27 are provided continuously with a region for firmly bonded connection (see hatched region) with the side edge of the respective corresponding second additional tab 24 to 27.

In contrast to the protective cover 1 of the fourth embodiment, the region of the firmly bonded connection in the region of the lower side edges of the first additional tabs 22, 23 is not configured continuously, but interruptedly. The protective cover 1 folded into the pocket shape thus has a total of three additional compartments. All three additional compartments are spatially separated from each other and from the main compartment.

FIG. 11A is a top view of the flat cut of the protective cover 1 from FIG. 10 with products arranged on it. More precisely, a medical product M is arranged on the base plate 2. A packing means P is arranged on the first additional tab 22, which is adjacent to the base plate 2. A medical product M is arranged on each of the second additional tabs 24, 25, which are adjacent to the first additional tab 22.

FIG. 11B is a top view of the flat cut formed into the protective cover 1 of FIG. 11A. In other words, the closed state of the pocket-shaped protective cover 1 of the fifth embodiment is shown in FIG. 11B. In this representation, the base plate 2 is facing the viewer. In the closed state, base plate 2 and cover plate 2 form the main compartment in which the medical product M is arranged. The packing means P is arranged in the additional compartment formed by the first additional tab 22 in cooperation with the first additional tab 23. In each case, a further medical product M is arranged in the additional compartment formed by the second additional tab 24 in cooperation with the second additional tab 26 and in the additional compartment formed by the second additional tab 25 in cooperation with the second additional tab 27.

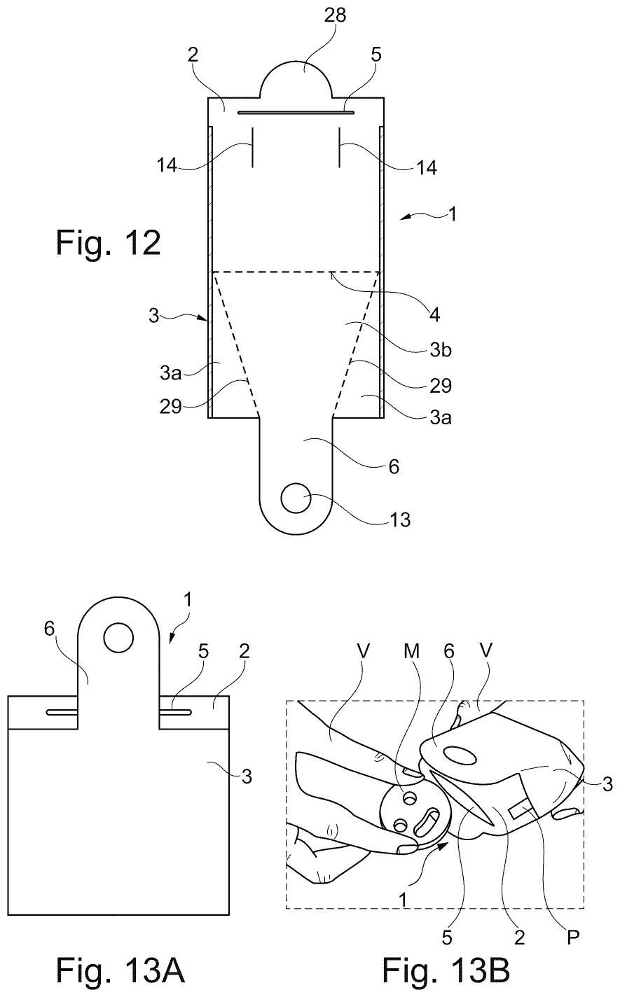

FIG. 12 is a top view of a flat cut of a protective cover 1 of a sixth embodiment. The flat cut of the protective cover 1 of the sixth embodiment is similar in its basic structure to the flat cut of the protective cover 1 according to the first embodiment. In particular, the differences between the flat cut of the protective cover 1 of the sixth embodiment and the flat cut of the protective cover 1 of the first embodiment are discussed below.

Here too, the short common side edge of base plate 2 and cover plate 3 is configured as a hinged fold line 4. From the other short side edge, in the immediate vicinity of which the receptacle 5 is arranged, a projection 28, in particular an arcuate one, extends outward. This can be gripped particularly easily by a user V, in particular when removing a medical product M, not shown here, from the flat cut folded into the pocket-shaped protective cover 1, thus facilitating the opening of the pocket-shaped protective cover 1 (see FIG. 14).

Furthermore, the cover plate 3 is subdivided into two portions: a connecting portion 3a and a tear-open portion 3b. The connecting portion 3a is divided into two halves by the tear-open portion 3b. Connecting portion 3a and tear-open portion 3b are separated from each other by two perforation lines 29. Connecting portion 3a and tear-open portion 3b are integrally configured with each other, with the perforation lines 29 defining a predetermined breaking point between the two portions 3a, 3b. The perforation lines 29 extend from the two opposite ends of the hinged fold line 4 and run conically or obliquely toward each other in the direction of the short side edge of the cover plate 3 opposite the hinged fold line 4. More precisely, the perforation lines 29 are configured linearly here. The perforation lines 29 are arranged or configured axially symmetrically to one another (in relation to a line bisecting the short side edges of the cover plate 3). The perforation lines 29 end at/open into the short side edge(s) of the cover plate 3 opposite the hinged fold line 4 and are spaced apart at this point by the width of the tab 6. The tab 6 of the cover plate 3 is integrally connected to the tear-open portion 3b. The tab 6 protrudes away from the cover plate 3 starting from the short side edge of the cover plate 3 opposite the hinged fold line 4.

The two halves of the connecting portion 3a each form the long side edges of the cover plate 3. The long side edges of the cover plate 3 run perpendicular to the hinged fold line 4 and adjoin it. The long side edges of the cover plate 3 have a region over their entire length via which the cover plate can be firmly bonded to the base plate 2, as shown by the hatched region. The base plate 2 also has a hatched region on its long side edges, which are perpendicular to the hinged fold line 4 and adjoin it, which is intended for firmly bonded connection to the corresponding region of the cover plate 3. The hatched region extends from the hinged fold line 4 in the direction of the short side edge of the base plate 2 opposite the hinged fold line 4. The hatched region of the base plate 2 is of the same length as the hatched region of the cover plate 3. The vertical distance between the two short side edges of the base plate is greater than the vertical distance between the two short side edges of the cover plate 3.

In this embodiment, the flat cut of the protective cover 1 also has in the base plate 2 the slits 14 for receiving a medical product or a packaging means accompanying the medical product, e.g. a drying agent, which run parallel to one another and are aligned perpendicular to the hinged fold line 4.

Advantageously, the flat cut of the protective cover 1 according to the sixth embodiment requires the least amount of material compared to the protective covers 1 of the first to fifth embodiments, since the flat cut of the protective cover 1 according to the sixth embodiment has no side flaps or additional tabs.

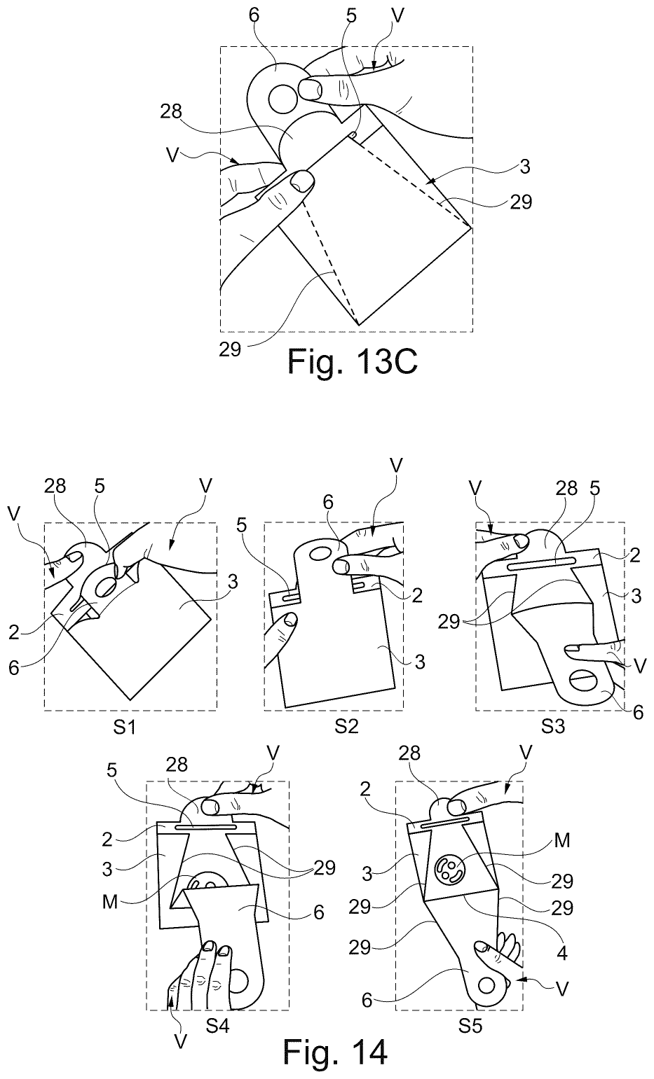

FIG. 13A shows a top view of a protective cover 1 folded from the flat cut of the protective cover 1 according to the sixth embodiment (see FIG. 12). The cover plate 3 is folded over the base plate 2 and the cover plate 3 and the base plate 2 are firmly bonded together in the respective hatched region (not shown here, see FIG. 12). The protective cover 1 shown in FIG. 13A thus forms a pocket that is open on one side.

FIG. 13B is a perspective view of the protective cover 1 of FIG. 13A, into which a medical product M (from a user V) is inserted. In contrast to the protective covers 1 of the first to fifth embodiments, the medical product M is only inserted into the protective cover 1 according to the sixth embodiment after the side edges of the base plate 2 and the cover plate 3 have been closed. A packing means P, for example a drying agent, is fixed in the slits 14.

FIG. 13C is a perspective view of the protective cover 1 of FIG. 13B, which is closed to the pocket shape and encloses the medical product M (and the packing means P). The protective cover 1 is closed in the region of the short side edges of the base plate 2 and the cover plate 3 opposite the hinged fold line 4. For this purpose, the tab 6 is inserted into the receptacle 5. The protective cover 1 shown in FIG. 13C is thus closed on all sides.

FIG. 14 is a pictorial representation of the process steps S1 to S5 for removing the medical product M from the protective cover 1 assembled according to FIG. 13A to FIG. 13C. In step S1, it is shown how the tab 6 is pulled out/removed from the receptacle 5 by a user V. In step S2, the user V grasps the tab 6 with one hand. In step S2, the user V grips the tab 6 with one hand. In step S3, the user V grips the projection 28 with another hand to fix the base plate 2. With one hand, the user V applies a pulling force to the tab 6 so that the tear-open portion 3b detaches from the connecting portion 3a along the perforation line 29. The connecting portion 3a is and remains firmly bonded to the base plate 2. In step S4, the tear-open portion 3b is already further separated from the connecting portion 3b compared to step S3, so that the medical product M is at least partially exposed from the tear-open portion 3b. In step S5, tear-open portion 3b and connecting portion 3a are completely separated from each other. The medical product M is completely exposed and can be removed from the protective cover 1 by the user V.

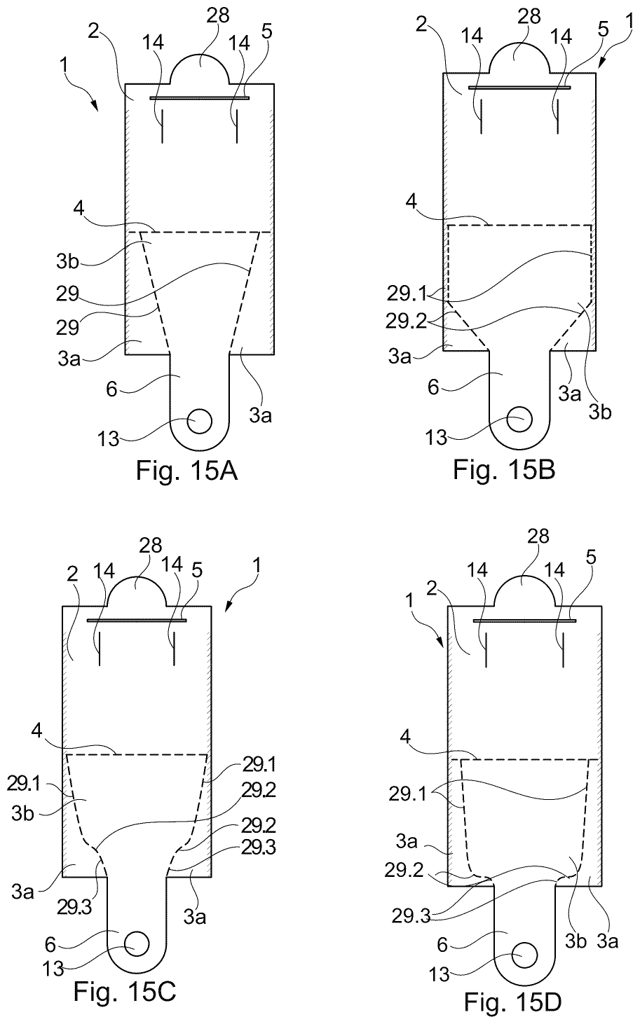

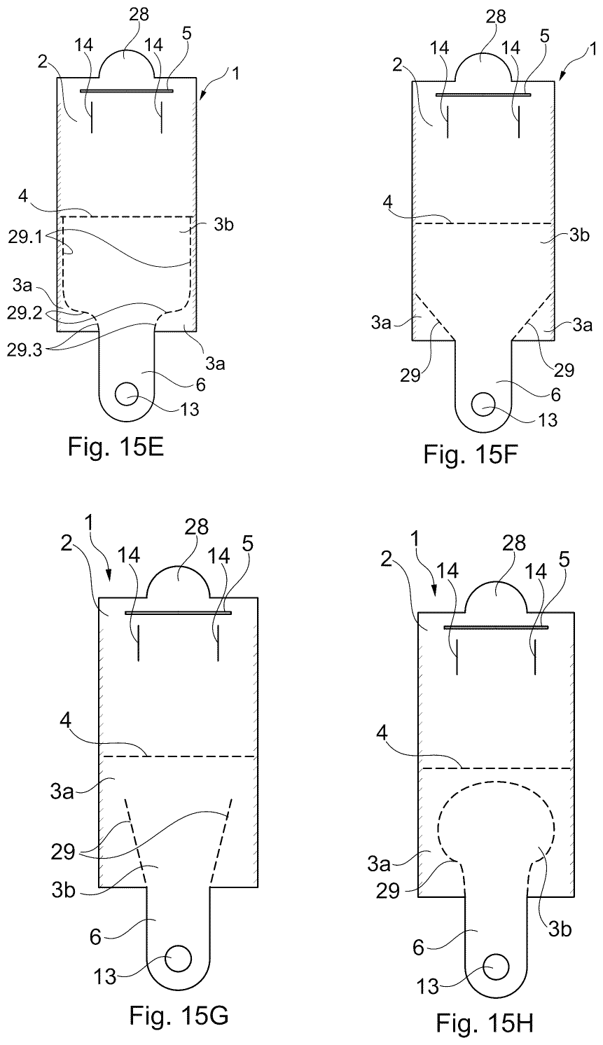

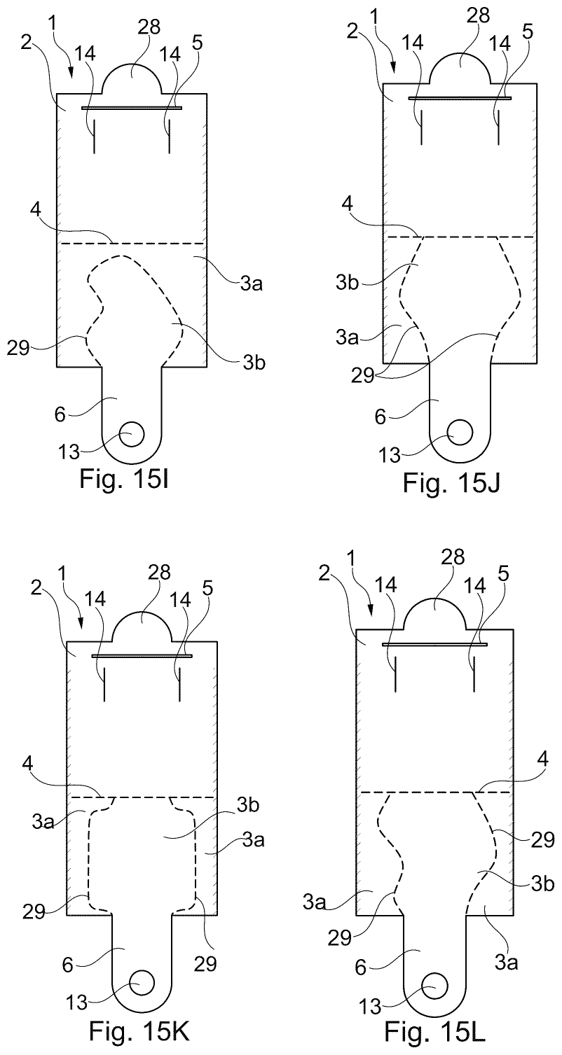

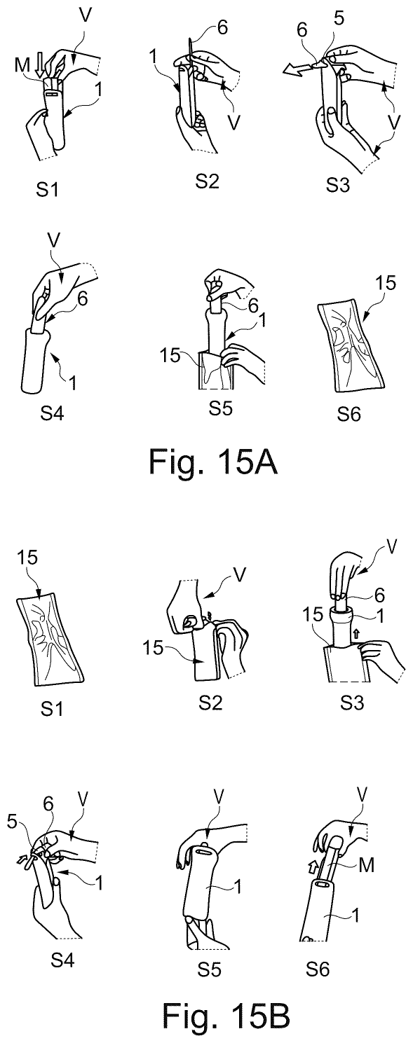

FIG. 15A is a plan view of a flat cut of a protective cover 1 of a first variant of the protective cover 1 according to the sixth embodiment. The flat cut of the protective cover 1 of the first variant is identical to the protective cover 1 of the sixth embodiment, apart from the difference described below.

In the protective cover 1 of the first variant, the two perforation lines 29 also end at the hinged fold line 4, but not at their opposite ends, but in each case at a distance from the opposite ends of the hinged fold line 4. Thus, in the protective cover 1 of the first variant, the area of the tear-open portion 3b defined between the perforation lines 29 is slightly smaller than the area of the tear-open portion 3b of the protective cover 1 according to the sixth embodiment.

FIG. 15B is a top view of a flat cut of a protective cover 1 of a second variant of the protective cover 1 according to the sixth embodiment. The flat cut of the protective cover 1 of the second variant is identical to the protective cover 1 of the sixth embodiment, apart from the difference described below.

In the protective cover 1 of the second variant, respective first (linear) perforation line portions 29.1 of the two perforation lines 29 extend from the opposite ends of the hinged fold line 4, initially substantially parallel to or along the long side edges of the cover plate 3 (up to over half the length of the cover plate 3). At this point, the first perforation line portions 29.1 end and are immediately/directly followed by second (linear) perforation line portions 29.2, which, starting from the connection point between the first and second perforation line portions 29.1, 29.2, extend obliquely in the direction of and up to the short side edge of the cover plate 3 opposite the hinged fold line 4. At this point, the second perforation line portions 29.2 are spaced apart by the width of the tab 6. In the case of the second variant of the protective cover 1 (and the fifth variant described later and shown in FIG. 15E), the first perforation line portions 29.1 directly adjoin the regions of the cover plate 3 (shown hatched) defining the long side edges of the cover plate 3, which are configured for a firmly bonded connection with the base plate 2. In other words, the first perforation line portions 29.1 are immediately adjacent or adjacent to the regions of the cover plate 3 configured for a firmly bonded connection to the base plate 2.

FIG. 15C is a top view of a flat cut of a protective cover 1 of a third variant of the protective cover 1 according to the sixth embodiment. The flat cut of the protective cover 1 of the third variant is identical to the protective cover 1 of the sixth embodiment, apart from the difference described below.

In the protective cover 1 of the third variant, the two perforation lines 29 are not configured linearly. Starting from the two opposite ends of the hinged fold line 4, the two first perforation line portions 29.1 extend in the direction of the short side edge of the cover plate 3 opposite the hinged fold line 4 and run conically or obliquely toward each other. The first perforation line portions 29.1 extend to just over half the length of the cover plate 3. The first perforation line portions 29.1 are immediately followed by second perforation line portions 29.2, which also extend inward at an angle and thus toward each other with a significantly flatter gradient than the first perforation line portions 29.1. The transition between the first and second perforation line portions 29.1 and 29.2 is configured as a rounded corner (non-linear!). The second perforation line portions 29.2 are immediately followed by third perforation line portions 29.3, which extend at a steeper incline than the second perforation line portions 29.2 diagonally inward and thus toward each other. The transition between the second and third perforation line portions 29.2 and 29.3 is configured as a rounded corner. The third perforation line portions 29.3 end at the short side edge of the cover plate 3 opposite the hinged fold line 4. At this point, the third perforation line portions 29.3 are spaced apart by the width of the tab 6. Preferably, the pitch of the third perforation line portions 29.3 corresponds to the pitch of the first perforation line portions 29.1.

FIG. 15D is a top view of a flat cut of a protective cover 1 of a fourth variant of the protective cover 1 according to the sixth embodiment. The flat cut of the protective cover 1 of the fourth variant is identical to the protective cover 1 of the third variant, apart from the difference described below.

Here, the perforation lines are configured non-linearly (with curves). In the protective cover 1 of the fourth variant, the first perforation line portions 29.1 each extend from a location lying on the hinged fold line 4, which is spaced apart from the opposite ends of the hinged fold line 4 (slightly, for example a few mm to a maximum of 2 cm, preferably a maximum of 1 cm). The first perforation line portions 29.1 run obliquely in the direction of the short side edge of the cover plate 3 opposite the hinged fold line 4 and thus run conically toward each other. The pitch of the first perforation line portions 29.1 of the fourth variant is steeper than the pitch of the first perforation line portions 29.1 of the third variant. Here, the first perforation line portions 29.1 extend to approximately ¾ of the length of the cover plate 3. The second perforation line portions 29.2 of the fourth variant have a smaller pitch than those of the third variant and run almost parallel to the hinged fold line 4. The third perforation line portions 29.3 of the fourth variant are significantly shorter (only a few mm to max. 2 cm, preferably max. 1 cm, long) than those of the third variant. At the point where the two perforation lines 29 open into the short side edge of the cover plate 3 opposite the hinged fold line 4, they are spaced apart by the width of the tab 6.

FIG. 15E is a top view of a flat cut of a protective cover 1 of a fifth variant of the protective cover 1 according to the sixth embodiment. The flat cut of the protective cover 1 of the fifth variant is identical to the protective cover 1 of the third variant, apart from the difference described below.

Here, the perforation lines are configured non-linearly (with bends/curves). In the protective cover 1 of the fifth variant, the first perforation line portions 29.1 extend from the opposite ends of the hinged fold line 4 parallel to or along the long side edges of the cover plate 3. The second perforation line portions 29.2 of the fifth variant have a smaller gradient than those of the third variant and run almost parallel to the hinged fold line 4. The third perforation line portions 29.3 of the fifth variant are slightly shorter than those of the third variant.

FIG. 15F is a plan view of a flat cut of a protective cover 1 of a sixth variant of the protective cover 1 according to the sixth embodiment. The flat cut of the protective cover 1 of the sixth variant is identical to the protective cover 1 of the sixth embodiment, apart from the difference described below.

In contrast to the protective cover 1 of the sixth embodiment and in contrast to the protective covers 1 of the first to fifth variants, the two (linear) perforation lines 29 of the sixth variant do not extend from the hinged fold line 4 to the short side edge of the cover plate 3 opposite the hinged fold line 4. The two perforation lines 29 of the sixth variant extend from (a point which, starting from the hinged fold line 4, lies above half the length of the cover plate 3, at) the long side edges of the cover plate 3 obliquely to the short side edge of the cover plate 3 opposite the hinged fold line 4. In other words, each perforation line 29 intersects in each case one of the long side edges of the cover plate 3 and the short side edge of the cover plate 3 opposite the hinged fold line 4.

FIG. 15G is a top view of a flat cut of a protective cover 1 of a seventh variant of the protective cover 1 according to the sixth embodiment. The flat cut of the protective cover 1 of the seventh variant is identical to the protective cover 1 of the first variant, apart from the difference described below.

In the protective cover 1, the (linear) perforation lines 29 do not adjoin the hinged fold line 4, but extend from a first location within the cover plate 3, which is at a (longitudinal) distance from the hinged fold line 4, (obliquely) to the short side edge of the cover plate 3 opposite the hinged fold line 4. The first location is spaced from the hinged fold line 4 by less than half the length of the cover plate 3.

FIG. 15H is a top view of a flat cut of a protective cover 1 of an eighth variant of the protective cover 1 according to the sixth embodiment. This protective cover 1 of the eighth variant differs from the protective cover 1 of the sixth embodiment and the protective cover 1 of the first to seventh variants in that only one continuous (non-linear) perforation line 29 is provided. The perforation line 29 is roughly mushroom-shaped, configured approximately in the manner of a stylized mushroom. It extends with a first end starting from the short side edge of the cover plate 3 opposite the hinged fold line 4 almost parallel to the long side edges of the cover plate 3. The perforation line 29 extends with a second end starting from the short side edge of the cover plate 3 opposite the hinged fold line 4 almost parallel to the long side edges of the cover plate 3 and almost parallel to the first end. The first and second ends of the perforation line 29 are spaced apart by the width of the tab 6. A mushroom-shaped line extends between the first and second ends of the perforation line 29. The perforation line 29 is arranged axially symmetrically (to a line bisecting the short side edges of the cover plate 3) on the cover plate 3.

FIG. 15I is a plan view of a flat cut of a protective cover 1 of a ninth variant of the protective cover 1 according to the sixth embodiment. As with the protective cover 1 of the eighth variant, the protective cover 1 of the ninth variant also has only one (continuous and non-linear) perforation line 29. Here, the perforation line 1 is not configured to be axially symmetrical. The perforation line 29 runs with several bends and curves. Both ends of the perforation line 29 lead into/end at the short side edge of the cover plate 3 opposite the hinged fold line 4 and are spaced apart at this point by the width of the tab 6.

FIG. 15J is a top view of a flat cut of a protective cover 1 of a tenth variant of the protective cover 1 according to the sixth embodiment. Here again, as in the protective cover 1 of the sixth embodiment and in the protective cover 1 of the first to seventh variants, two (non-linear) perforation lines 29 are provided. These each extend at a distance from the opposite ends of the hinged fold line 4, starting from the hinged fold line 4 to the short side edge of the cover plate 3 opposite the hinged fold line 4. The two perforation lines 29 together form approximately a bulbous vase shape. The perforation lines 29 are configured axially symmetrically to each other (in relation to a line bisecting the short side edges of the cover plate 3). At the point where the two perforation lines 29 open into the short side edge of the cover plate 3 opposite the hinged fold line 4, they are spaced apart by the width of the tab 6.

FIG. 15K is a plan view of a flat cut of a protective cover 1 of an eleventh variant of the protective cover 1 according to the sixth embodiment. The (mainly linear, partly non-linear) perforation lines 29 of the protective cover 1 of the eleventh variant extend similarly to the perforation lines of the protective cover 1 of the tenth variant. However, the two perforation lines 29 do not form a bulbous vase shape here, but run largely/mainly parallel to or along the long side edges of the cover plate 3. The perforation lines 29 are spaced apart from the long side edges of the cover plate 3 in each case. The perforation lines 29 are configured axially symmetrically to each other (in relation to a line bisecting the short side edges of the cover plate 3). At the point where the two perforation lines 29 open into the short side edge of the cover plate 3 opposite the hinged fold line 4, they are spaced apart by the width of the tab 6.

FIG. 15L is a top view of a flat cut of a protective cover 1 of a twelfth variant of the protective cover 1 according to the sixth embodiment. Here again, two (non-linear) perforation lines 29 are provided. The two perforation lines 29 run in a curved or wavy shape. The two perforation lines 29 run with lines that are curved differently to each other and are not configured symmetrically to each other. At the point where the two perforation lines 29 open into the short side edge of the cover plate 3 opposite the hinged fold line 4, they are spaced apart by the width of the tab 6.

FIG. 16A schematically shows the process steps S1 to S7 for inserting a medical product M into a packaging according to the disclosure with pocket-shaped protective cover 1 and sterile packaging 15. In step S1, the medical product M is first inserted into the protective cover 1, which has already been folded into the pocket shape. In step S2, the tab 6 of the protective cover 1, in which the medical product M is arranged, is gripped by the user V. In step S3, the tab 6 on base plate 2 or cover plate 3 is inserted into the corresponding receptacle 5 on the other of base plate 2 or cover plate 3. In step S4, the tab 6 is pulled completely straight and the user V grips the protective cover 1 on the tab 6. In step S5, the protective cover 1 is inserted into the sterile packaging 15. The protective cover 1 is held and guided by the tab 6. The protective cover 1 is positioned in the sterile packaging 15 in such a way that it is completely accommodated by the sterile packaging 15. In step S6, the sterile packaging 15 with the protective cover 1 arranged therein, in which the medical product M is again arranged, is vacuumed. After step S6, the packaging system is ready for sterile transportation.

FIG. 16B shows the process steps S1 to S6 for unpacking the medical product M from the packaging according to the disclosure. In step S1, the vacuumed sterile packaging 15 is shown again, in which the protective cover 1 is arranged, in which in turn the medical product M is arranged (as in S6 of FIG. 15A). In step S2, the sterile packaging 15 is opened or torn open manually, in particular at a perforated point. In step S3, the protective cover 1 is pulled out of the sterile packaging 15. The protective cover 1 is gripped by the tab 6. In step S4, the tab 6 is pulled out of the receptacle 5. In step S5, the user V grips the protective cover 1, which is still folded into pocket shape here, in order to grasp the medical product M arranged in the protective cover 1. In step S6, the user V pulls the medical product M out of the protective cover 1.

When it is described in this application that the perforation lines 29 extend to or from the hinged fold line 4, this includes the case that the perforation lines 29 end or begin in the vicinity of the hinged fold line 4, i.e. up to 1 cm, preferably up to 0.5 cm, away from the hinged fold line 4. This applies in particular to the embodiments of FIGS. 12 to 14, 15A to 15F and 15J to 15L.

The aspects of the various embodiments can be combined with each other as long as this is technically possible and useful.

### LIST OF REFERENCE SIGNS

1 protective cover
2 base plate
3 cover plate
3a connecting portion of the cover plate
3b tear-open portion of the cover plate
4 hinged fold line (between base and cover plate)
5 receptacle
6 tab
7 first side plate
8 second side plate
9 swivel fold line (between base plate and first side plate 7)
10 swivel fold line (between base plate and second side plate 8)
11 latching slit in first side plate 7
12 latching slit in second side plate 8
13 recess
14 slits in base plate
15 sterile packaging or secondary packaging
16 (slit-like) receptacle in first side plate 7
17 (slit-like) receptacle in second side plate 8
18 closing tab
19 barbed hook
20 insert tab
21 tab receptacle
22, 23 first additional tabs
24 to 27 second additional tabs
28 projection
29 perforation line
29.1-29.3 first to third perforation line portion
M medical product
P packing means
V user

1. A packaging for or of a medical product, the packaging comprising:
a secondary packaging forming a sterile barrier, the sterile barrier forming a sealed or sealable receiving space for the medical product; and
a product storage space and/or holder for contact-free storage of the medical product within the secondary packaging,
the product storage space and/or holder being a protective cover for receiving the medical product and for interspacing the secondary packaging of the medical product, which is made of a single, flexible flat cut by folding along at least one hinged-fold line.

2. The packaging according to claim 1, wherein the protective cover is closed on all sides toward the secondary packaging in a closed state produced by folding.

3. The packaging according to claim 1, wherein the flat cut comprises:
a base plate serving as a storage space for the medical product and having a base latching device;
a folding or cover plate coupled to the base plate via the hinged-fold line, the folding or cover plate being operatively engageable with the base latching device in a position where the folding or cover plate is folded toward/onto the base plate to hold a folded position; and
a first side plate is coupled to the base plate via a swivel fold line aligned perpendicularly to the hinged-fold line and has, the first side plate comprising a first side latching device for holding the first side plate in a first position pivoted toward the base plate.

4. The packaging according to claim 3, wherein
the flat cut further comprises a second side plate coupled to the base plate opposite the first side plate via a swivel fold line aligned perpendicularly to the hinged-fold line, the second side plate comprising a second side latching device for holding the second side plate in a second position pivoted toward the base plate.

5. The packaging according to claim 4, wherein:
the first side plate comprises a first side engagement device,
the second side plate comprises a second side engagement device,
and wherein:
the first side engagement device and the second side plate engagement device are each configured as a first latching slit and a second latching slit, respectively, wherein the first side plate and the second side plate are configured to hook together via the first latching slit and the second latching slit, or
the first side engagement device and the second side engagement device are each configured as a receptacle into which a tab-like portion of the folding or cover plate is insertable.

6. The packaging according to claim 1, wherein the flat cut comprises:
a base plate serving as a storage space for the medical product and having a base latching device;
a folding or cover plate coupled to the base plate via the hinged-fold line, the folding or cover plate operatively engageable with the base latching device in a position where the folding or cover plate is folded toward/onto the base plate in order to hold a folded position;
a closing tab coupled to the base plate or the folding or cover plate via a swivel fold line aligned parallel to the hinged-fold line; and
an insert tab coupled via a swivel fold line aligned parallel to the hinged-fold line to that of the base plate and folding or cover plate which is not coupled to the closing tab, the packaging comprising a tab receptacle between the insert tab and the base plate or folding or cover plate coupled thereto, the tab receptacle configured to receive the closing tab.

7. The packaging according to claim 2, wherein the flat cut comprises:
a base plate serving as a storage space for the medical product and having a base latching device;
a folding or cover plate coupled to the base plate via the hinged-fold line, the folding or cover plate operatively engageable with the base latching device in a position where the cover plate is folded toward/onto the base plate to hold a folded position; and
in each case a first additional tab on the base plate and on the folding or cover plate, which adjoin side edges of the base plate and of the folding or cover plate which are adjacent to one another and extend perpendicularly to the hinged-fold line and, in the closed state, together form an additional compartment for receiving a further product.

8. The packaging according to claim 7, wherein the flat cut furthermore has further second additional tabs on the base plate and on the folding or cover plate, which in each case adjoin a side edge of the first additional tab, and the second additional tabs on the base plate or the folding or cover plate are spaced apart from one another and, in the closed state, together with the second additional tab lying above or below it, form further additional compartments for receiving further products on the other of the base plate and the folding or cover plate.

9. The packaging according to claim 1, wherein the flat cut comprises:
a base plate serving as a storage space for the medical product and having a base latching device; and
a folding or cover plate, which is coupled to the base plate via the hinged-fold line, the folding or cover plate operatively engageable with the base latching device in a position where the folding or cover plate is folded toward/onto the base plate in order to hold the folded position,
the folding or cover plate being divided into a connecting portion and a tear-open portion separated from the connecting portion by at least one perforation line that forms a predetermined breaking point between the connecting portion and the tear-open portion.

10. The packaging according to claim 9, wherein a first end of the at least one perforation line and a second end of said at least one perforation line or, where the at least one perforation line comprises two perforation lines on the folding or cover plate, a first perforation line and a second perforation line open into a side edge of the folding or cover plate opposite the hinged-fold line.

11. The packaging according to claim 10, wherein, starting from the side edge of the folding or cover plate opposite the hinged-fold line, a tab-like portion of the folding or cover plate extends away from the folding or cover plate, which is operatively engageable with the base latching device on the base plate and integrally configured with the tear-open portion.

12. The packaging according to claim 11, wherein the first end of the at least one perforation line and the second end of said at least one perforation line or, where the at least one perforation line comprises two perforation lines on the folding or cover plate, the first perforation line and the second perforation line are spaced apart by a width of the tab-like portion at a point at which they each open into the side edge of the folding or cover plate opposite the hinged-fold line.

13. The packaging according to claim 9, wherein two perforation lines are provided which are configured non-linearly and are arranged axially symmetrically to one another with respect to a line bisecting the hinged-fold line and extend continuously in each case starting from the hinged-fold line the the to a side edge of the folding or cover plate opposite the hinged-fold line.

14. The packaging according to claim 2, wherein a base latching device of a base plate is configured as a receptacle, which is arranged in proximity to a side edge of the base plate, and a folding or cover plate has a tab-shaped portion which extends outwardly from the side edge of the folding or cover plate, which in the closed state is opposite the side edge with receptacle of the base plate, and which is insertable into the receptacle of the base plate, or
the base latching device of the base plate is configured as a tab-shaped portion of the base plate and the folding or cover plate has a receptacle which receives the tab-shaped portion of the base plate in the closed state.

15. The packaging according to claim 1, wherein the protective cover or the flat cut forming the protective cover is made of a flexible, abrasion-resistant and sterilizable material.

16. The packaging according to claim 1, wherein the protective cover is shaped like a pocket.

17. The packaging according to claim 5, wherein the receptacle forming the engagement device of the first and second side plate is formed like a slit.

18. The packaging according to claim 6, wherein the tab receptacle is formed like a slit.

19. The packaging according to claim 14, wherein the receptacle forming the base latching device is formed like a slit.
